(19) United States
(12) Patent Application Publication
(10) Pub. No.: US 2018/0116636 A1
(43) Pub. Date: May 3, 2018

(54) ULTRASONIC IMAGE GENERATION SYSTEM

(71) Applicant: SOCIONEXT INC., Yokohama-shi (JP)

(72) Inventors: Naoto YONEDA, Yokohama-shi (JP); Naoto Adachi, Yokohama-shi (JP); Hiroaki Takagi, Yokohama-shi (JP); Masaya Tamamura, Yokohama-shi (JP); Amane Inoue, Yokohama-shi (JP)

(21) Appl. No.: 15/860,086

(22) Filed: Jan. 2, 2018

Related U.S. Application Data
(63) Continuation of application No. PCT/JP2016/056093, filed on Feb. 29, 2016.

(30) Foreign Application Priority Data
Jul. 9, 2015 (JP) 2015-138059

(51) Int. Cl. A61B 8/08; A61B 8/00

(52) U.S. Cl. CPC A61B 8/5207; A61B 8/462; A61B 8/4472

(57) ABSTRACT

An ultrasonic image generation system having an ultrasonic unit configured to transmit and receive an ultrasonic signal, a drive control/signal processing unit configured to repeat processing to generate an ultrasonic image signal by processing a received signal of the ultrasonic unit as well as to generate a drive signal that is supplied to the ultrasonic unit; and a display unit configured to repeat displaying an ultrasonic image based on the ultrasonic image signal, to stop updating of a displayed image in response to a stop signal input, and to resume updating of the displayed image in response to a start signal input, wherein the drive control/signal processing unit stops at least part of an operation in response to the stop signal input and resumes the stopped operation in response to the start signal input.

### CROSS-REFERENCE TO RELATED APPLICATION

This application is a continuation application of International Application PCT/JP2016/056093 filed on Feb. 29, 2016, the entire contents of which are incorporated herein by reference.

### FIELD

The present invention relates to an ultrasonic image generation system.

### BACKGROUND

An ultrasonic image generation system that generates an ultrasonic image representing a state of the inside of a living body by irradiating the living body with an ultrasonic wave and detecting the reflected wave is widely used. A common ultrasonic image generation system has a main body unit and an ultrasonic transducer (hereinafter, referred to as an ultrasonic unit) connected to the main body unit by a cable. The main body unit generates a drive signal of the ultrasonic unit and transmits the generated drive signal to the ultrasonic unit via the cable. The ultrasonic unit outputs an ultrasonic wave in accordance with the drive signal, generates a reflected ultrasonic wave signal by capturing the reflected ultrasonic wave, and transmits the reflected ultrasonic wave signal to the main body unit. The main body unit generates an ultrasonic image by processing the received reflected ultrasonic wave signal and displays the ultrasonic image on a display.

The main body unit repeats the generation of a drive signal that is necessary to generate ultrasonic images corresponding to one screen and in response to the generation of the drive signal, the generation of ultrasonic image signals corresponding to one screen is repeated and an ultrasonic image is displayed real time. An observer observes the ultrasonic image that changes real time, and when the observer observes particularly, the observer performs an operation to give instructions to hold image updating. In response to the operation, the displayed ultrasonic image is maintained without updated, and the observer observes the fixed ultrasonic image in detail and performs a necessary task, such as measurement, and if necessary, the observer gives instruction to store the ultrasonic image in a storage device. When instructions to start ultrasonic image updating are given in the state where ultrasonic image updating is held, ultrasonic image updating is resumed.

Even while the ultrasonic image updating is held, the generation of the drive signal and the generation of the ultrasonic image signal are repeated, from the viewpoint of operability that the image is immediately updated to a new image when the displayed image updating is resumed.

In recent years, an ultrasonic image generation system is expected to be turned into a mobile device and reduction in size, reduction in cost, and improvement of operability have been sought. Thus, it has been proposed that the portion relating to the drive signal generation of the ultrasonic unit and the reflected ultrasonic wave signal processing in the main body unit is turned into a compact probe in accordance with the ultrasonic unit, a wireless communication function is installed in the probe, and the probe and the display unit configured to display an ultrasonic image are connected by wireless communication. Thus, the probe is a wireless probe and operability improves and, if a general-purpose communication terminal having a display function as a display unit is used, an ultrasonic image generation system may be implemented at a low cost.

### RELATED DOCUMENTS

[Patent Document 1] Japanese National Publication of International Patent Application No. 2002-530174

[Patent Document 2] Japanese Laid Open Patent Document No. 2008-61938

### SUMMARY

An ultrasonic image generation system of a first aspect has an ultrasonic unit configured to transmit and receive an ultrasonic signal, a drive control/signal processing unit, and a display unit. The drive control/signal processing unit repeats processing to generate an ultrasonic image signal by processing a received signal of the ultrasonic unit as well as to generate a drive signal that is supplied to the ultrasonic unit. The display unit repeats displaying an ultrasonic image based on the ultrasonic image signal, holds displayed image updating in accordance with a stop signal input, and resumes displayed image updating in accordance with a start signal input. The drive control/signal processing unit holds at least part of the operation in accordance with the stop signal input and resumes the held operation in accordance with the start signal input.

The object and advantages of the embodiments will be realized and attained by means of the elements and combination particularly pointed out in the claims.

It is to be understood that both the foregoing general description and the following detailed description are exemplary and explanatory and are not restrictive of the invention.

### BRIEF DESCRIPTION OF DRAWINGS

FIG. 1 is a block diagram illustrating a configuration of an ultrasonic image generation system of an embodiment.

FIG. 2A and FIG. 2B are each a diagram illustrating a configuration for a user to perform an operation input in the ultrasonic image generation system of the embodiment, and FIG. 2A illustrates switch arrangement on the side of a wireless probe and FIG. 2B illustrates a display example on the side of a tablet.

FIG. 3 is an operation flowchart of the ultrasonic image generation system of the embodiment.

FIG. 4 is a diagram illustrating a configuration of the ultrasonic image generation system of the embodiment in FIG. 1 in more detail, and, explaining blocks that turn off in a measurement stop (idle) state.

FIG. 5 is a diagram illustrating a configuration of a clock control unit.

FIG. 6 is a diagram illustrating a table of combination examples of blocks that power down.

### DESCRIPTION OF EMBODIMENTS

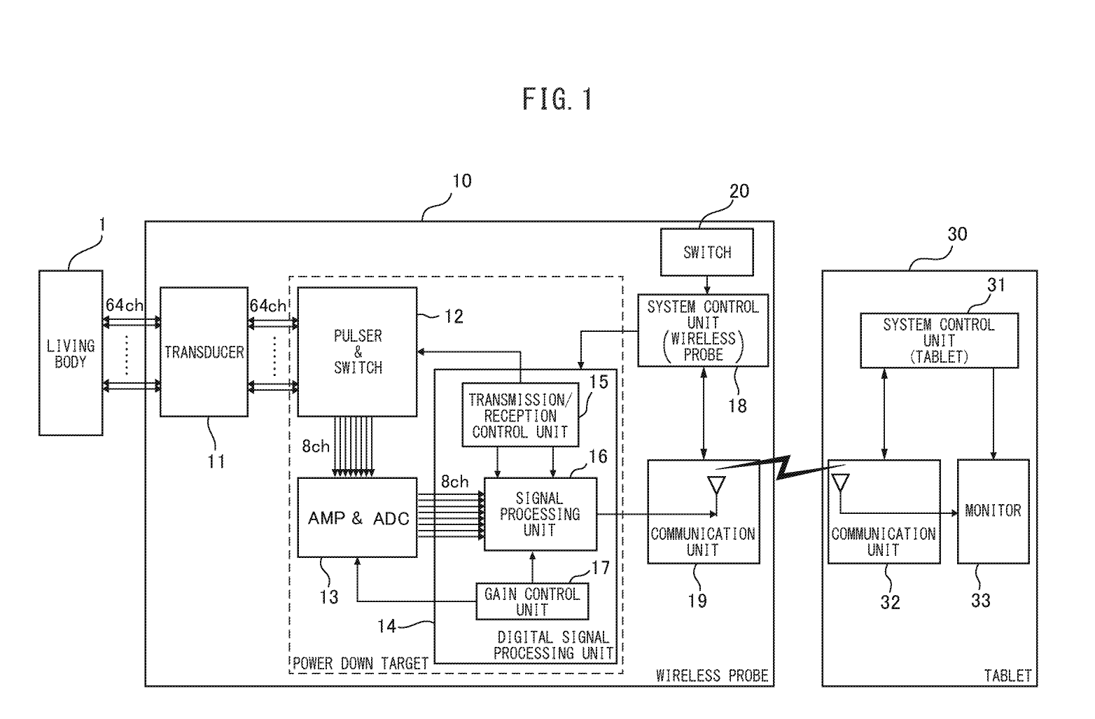

FIG. 1 is a block diagram illustrating a configuration of an ultrasonic image generation system of an embodiment.

The ultrasonic image generation system of the embodiment has a wireless probe 10 and a tablet 30. The wireless probe 10 is held by a user of the ultrasonic image generation system and contacts with the surface of a living body 1, which is the target of measurement, and the inside of the living body 1 is measured by ultrasonic waves. As the tablet 30, a widely spread PC tablet is used by installing application software for an ultrasonic image generation system. The wireless probe 10 and the tablet 30 each have a short-range wireless communication function (for example, BLUETOOTH (registered trademark) and are capable of wirelessly communicating (wireless communication) with each other. The tablet 30 is not limited to a PC tablet, and a PC, a smartphone having a wireless communication function may be used.

The wireless probe 10 has a transducer 11, a pulser & switch 12, an AMP & ADC 13, a digital signal processing unit 14, a (wireless probe) system control unit 18, a communication unit 19, and a switch 20. The digital signal processing unit 14 has a transmission/reception control unit 15, a signal processing unit 16, and a gain control unit 17. Although not illustrated schematically, the wireless probe 10 has a battery and each unit is driven by a battery. The battery may be a primary battery or a rechargeable secondary battery.

The transducer 11 converts a high-voltage pulse signal from the pulser & switch 12 into a sound wave, outputs the sound wave to the living body 1, and converts the sound wave reflected at the boundary between muscle and fat and the like within the living body 1, whose acoustic impedances are different, into an electric signal. The pulser & switch 12 selects the electric signal of the reflected sound wave by a switch circuit and outputs the electric signal to the AMP & ADC 13. The pulser & switch circuit 12 performs processing to bring into focus the signals transmitted simultaneously in a plurality of channels within the living body by changing the amount of delay for each channel. After amplifying the electric signal by the amplifier (AMP), the AMP & ADC 13 converts the electric signal into a digital signal by the ADC (Analog-to-Digital Converter) and outputs the digital signal to the digital signal processing unit 14. Here, an example is illustrated, in which the number of input/output channels of the transducer 11 is sixty-four and the number of channels of the AMP & ADC 13 is eight, but these numbers are optional and the width and resolution of an ultrasonic image that is obtained are determined by the channel interval and the number of channels of the transducer 11. The pulser & switch 12 performs simultaneous pulser transmission corresponding to the number of channels of the ADC. The configuration of the above-described portion is described in Patent Documents 1, 2, and is known widely, and therefore explanation thereof is omitted.

In the digital signal processing unit 14, the transmission/reception control unit 15 controls the focusing processing by the above-described pulser & switch circuit 12. The signal processing unit 16 converts the digital signal from the AMP & ADC 13 into luminance information by using the control signal from the transmission/reception control unit 15. The gain control unit 17 performs gain correction or the like that considers the attenuation within the living body. By the above processing, an ultrasonic image can be obtained by performing transmission and reception while shifting the sixty-four channels one by one and processing the received signals.

The communication unit 19 of the wireless probe 10 performs short-range wireless communication to receive an operation command and the like from the tablet 30 and outputs the command to the system control unit 18 and, transmits the ultrasonic image data generated by the digital signal processing unit 14 to the tablet 30. The system control unit 18 controls the entire wireless probe 10 based on the operation command and the like from the communication unit 19 and the operation signal of the switch 20. The switch 20 is a button switch provided to the wireless probe 10 and the operation of the switch 20 will be described later.

The portion except for the system control unit 18 enters the off state when the power source is turned off. It is necessary for the system control unit 18 to detect the operation of the switch 20 to turn on the power source, and therefore the system control unit 18 remains on even while the power source is off. The portion unrelated to the detection of the operation of the switch 20 within the system control unit 18 may be turned off when the power source is turned off.

The tablet 30 has a (tablet) system control unit 31, a communication unit 32, and a monitor 33. The system control unit 31 performs control of the tablet. The communication unit 32 performs short-range wireless communication and transmits an operation command and the like to the wireless probe 10 and, receives ultrasonic image data from the wireless probe 10. The monitor 33 is a display device having a touch screen function and displays ultrasonic image data and, detects the operation by a user, which is input by using the touch screen function. The monitor 33 is not limited to one having the touch screen function and may be one having a display unit, an operation switch. The capacity of the power source of the tablet 30 is sufficiently large and power consumption does not bring any problem, and therefore explanation thereof is not given in particular.

In the ultrasonic image generation system of the embodiment, a power down control unit is provided to the system control unit 18, in addition to the above-described configuration, and therefore i power down control may be carried out in accordance with a switch operation.

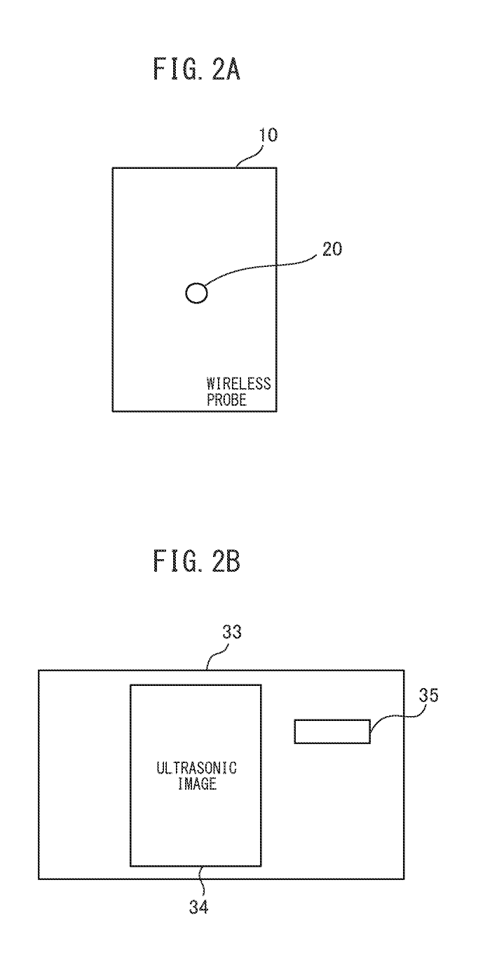

FIG. 2A and FIG. 2B are each a diagram illustrating a configuration for a user to perform an operation input in the ultrasonic image generation system of the embodiment, and FIG. 2A illustrates switch arrangement on the side of the wireless probe and FIG. 2B illustrates a display example on the side of the tablet.

As illustrated in FIG. 2A, the wireless probe 10 has the shape of a substantially rectangular parallelepiped. For example, on the lower side in FIG. 2A, the transducer 11 is provided and the bottom surface contacts with the living body 1 and sound waves are input and output at the bottom surface. The button switch 20 for operation is arranged on the lateral side. A user grasps the lateral side of the wireless probe 10 by hooking his/her finger on the button switch 20, contacts the bottom surface with the living body 1, and operates the button switch 20. The operation to press down the button switch 20 in the state where the power source of the wireless probe 10 is off is determined to be the operation to turn on the power source of the wireless probe 10. The operation to press down the button switch 20 for a short time in the state where the power source is on and the image is held (measurement stop) is determined to be instructions to change the state into the state where the image display is updated (during measurement) and the operation to press down the button switch 20 for a predetermined time or longer is determined to be the operation to change the power source of the wireless probe 10 into the off state. Hereinafter, the operation to press down the button switch 20 for a short time is referred to as short pressing and the operation to press down the button switch 20 for a predetermined time or longer is referred to as long pressing. Further, the short pressing operation in the state where the power source is on and the image is updated (during measurement) is determined to be the operation to change the state into the state where the image is held (measurement stop) and the long pressing operation is determined to be the operation to change the power source of the wireless probe 10 into the off state.

As shown in FIG. 2B, on the monitor 33 of the tablet 30, an ultrasonic image 34 and an operation button 35 by the touch screen function are displayed and various displays are produced other than the ultrasonic image 34 and the operation button 35, but they are not illustrated schematically. While the ultrasonic image 34 is updated real time, “stop” is displayed on the operation button 35 and when “stop” is touched, the ultrasonic image 34 is held (updating of the image display is held and the image when “stop” is touched continues to be displayed) (stop). Further, while the ultrasonic image 34 is held (measurement stop), “start” is displayed on the operation button 35 and when “start” is touched, updating of the ultrasonic image 34 is resumed (start).

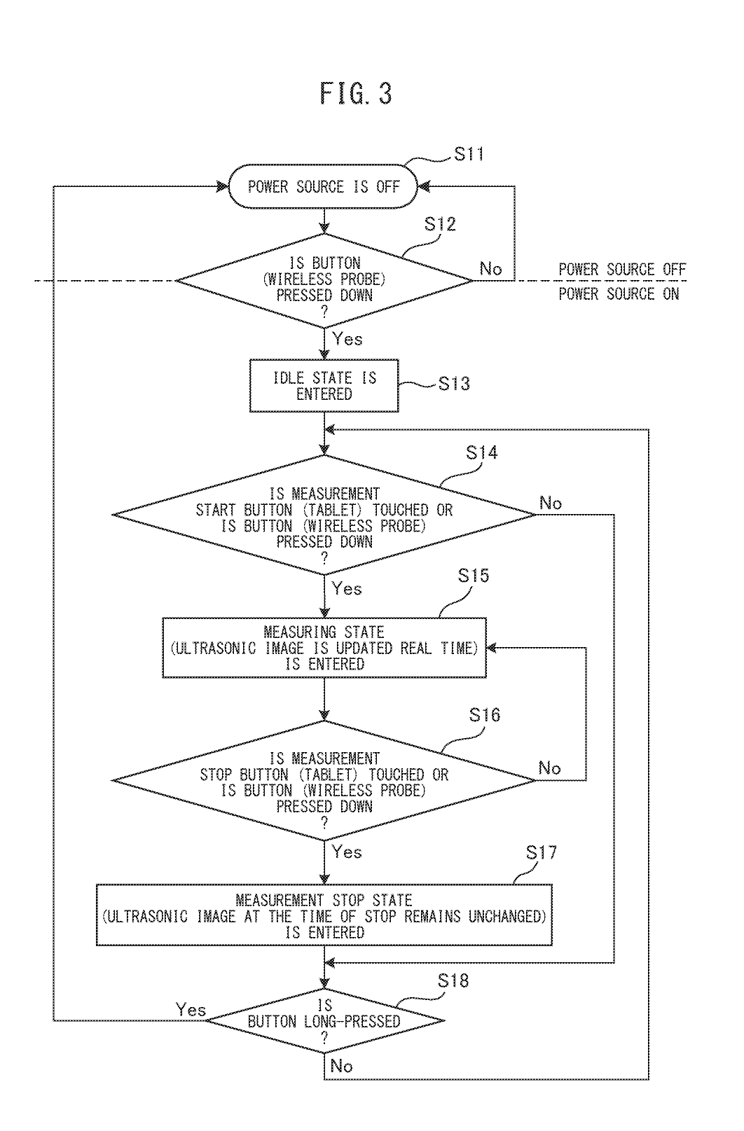

FIG. 3 is an operation flowchart of the ultrasonic image generation system of the embodiment.

As illustrated schematically, the operation flow is divided into the state where the power source of the wireless probe 10 is ON and the state where the power source is OFF. The flow is illustrated on the assumption that the tablet 30 is in the power source ON state.

At step S11, the wireless probe 10 is in the power source OFF state.

At step S12, whether the button switch 20 is pressed down is detected and when the pressing down is not detected, the processing returns to step S11 and when the pressing down is detected, the wireless probe 10 is brought into the power source ON state and the processing advances to step S13.

At step S13, the wireless probe 10 enters the idle state. The idle (stop) state is the same as a measurement stop state, described later, in that image updating is not carried out, but the immediately previous ultrasonic image does not exist, and therefore no ultrasonic image is displayed on the monitor 33. In the idle (stop) state, part of the portion surrounded by the broken line, which is described as “power down target”, in FIG. 1 of the wireless probe 10 is in the OFF state.

At step S14, whether the operation button (start) 35 is touched on the monitor 33 of the tablet 30, or the button switch 20 is pressed down is detected. When the touch and the pressing down are not detected, the processing advances to step S18 and when the touch or the pressing down is detected, the processing advances to step S15.

At step S15, the wireless probe 10 enters the measuring state and all the portions of the wireless probe 10 enter the ON state, and the ultrasonic image is updated real time.

At step S16, whether the operation button (stop) 35 is touched on the monitor 33 of the tablet 30, or the button switch 20 is pressed down is detected. When the touch and the pressing down are not detected, the processing returns to step S15 and when the touch or the pressing down is detected, the processing advances to step S17.

At step S17, the wireless probe 10 enters the measurement stop state and the ultrasonic image displayed on the monitor 33 when the wireless probe 10 is stopped is not updated and remains unchanged. At this time, as will be described later, part of the portion surrounded by the broken line, which is described as “power down target”, in FIG. 1 of the wireless probe 10 is in the OFF state.

At step S18, whether the button switch 20 is long-pressed is determined and when the button switch 20 is long-pressed, the power source of the wireless probe 10 is turned off and the processing returns to step S11 and when the button switch 20 is not long-pressed, the processing returns to step S14.

As explained above, the ultrasonic image generation system of the embodiment is roughly divided into two states, one is the state where the power source of the wireless probe 10 is OFF and the other is the state where the power source is ON. The power source ON state includes two states, one is the measurement stop (idle) state and the other is the measuring state. As described above, in the ultrasonic measurement, it is necessary to fix the ultrasonic image once, which is updated real time during measurement, by bring about the measurement stop state. Thus, the measuring state and the measurement stop state are repeated at the time of actual use. During measurement, it is necessary to acquire and update the ultrasonic image at all times, and therefore it is difficult to power down a specific block, but in the measurement stop state, it is not necessary to acquire the ultrasonic image, and therefore each block may be powered down.

Next, the portion that turns off in the measurement stop (idle) state in the wireless probe 10 is explained.

The blocks that turn off in the measurement stop state are the pulser & switch 12, the AMP & ADC 13, and the digital signal processing unit 14 within “power down target” surrounded by the broken line in FIG. 1. The system control unit 18 and the communication unit 19 need to receive the notification of the pressing down detection of the button switch 20 and the button (start, stop) operation of the tablet 30 at all times, and therefore they are excluded from the power down target.

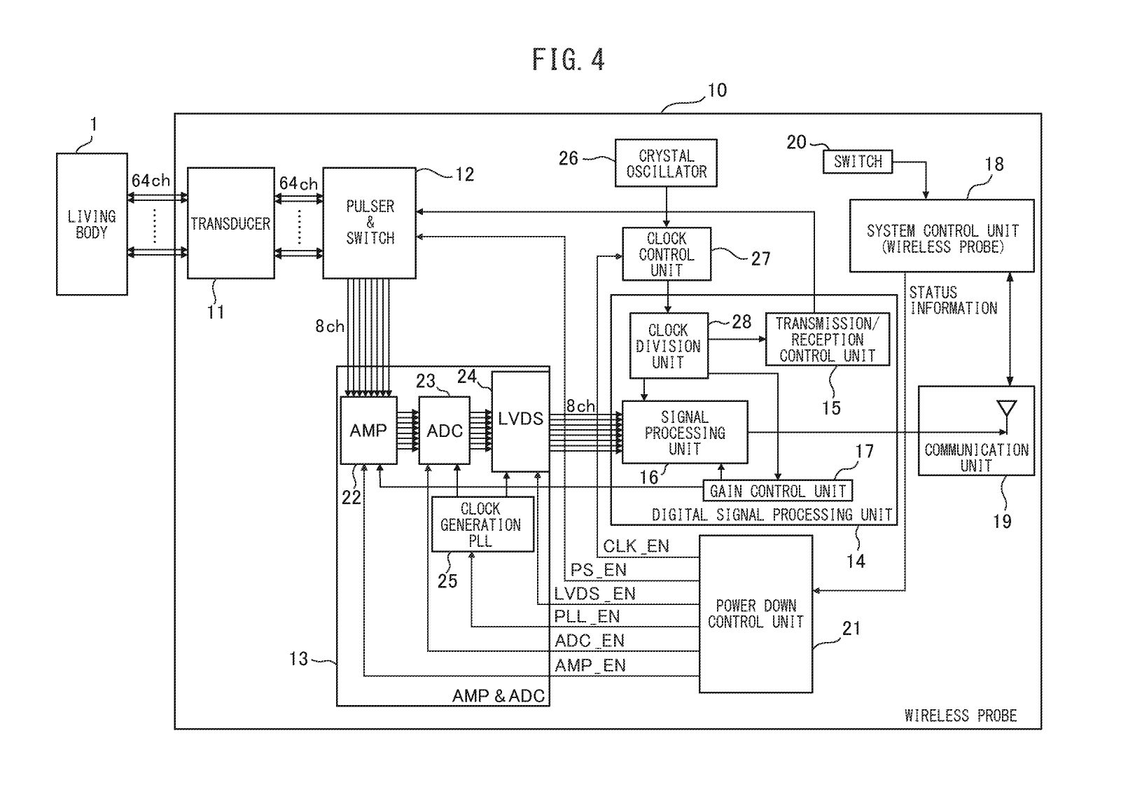

FIG. 4 is a diagram explaining the blocks that turn off in the measurement stop (idle) state, as well as illustrating in more detail the configuration of the ultrasonic image generation system of the embodiment in FIG. 1.

In FIG. 4, a power down control unit 21, a crystal oscillator 26, and a clock control unit 27 are further illustrated. The power down control unit 21 and the clock control unit 27 are portions that are originally included in the system control unit 18, but in order to make easy the schematic representation, in FIG. 4, they are illustrated outside the system control unit 18. Further, the schematic representation of the crystal oscillator 26 is omitted in FIG. 1.

The power down control unit 21 performs power down control based on status information from the system control unit 18. The status information is information to determine whether the state is the measuring state or the measurement stop state and the power down control unit 21 performs control so as to keep the power down state during measurement stop and releases the power down state during measurement.

The crystal oscillator 26 generates and outputs a clock based on which the wireless probe 10 performs the clock operation. The clock control unit 27 controls whether to perform or stop the clock supply in the digital signal processing unit 14 from the crystal oscillator 26.

As illustrated schematically, the AMP & ADC 13 has an AMP (amplifier) 22, an ADC 23, an LVDS (Low Voltage Differential Signaling) 24, and a clock generation PLL 25. The AMP 22 amplifies each 8-ch signal from the pulser & switch 12. The ADC 23 converts each 8-ch output signal of the AMP 22 into a digital signal. The LVDS 24 converts the 8-ch digital signal (parallel signal) output from the AMP 22 into a low voltage differential signal (serial signal) to be transmitted to the digital signal processing unit 14 and outputs the low voltage differential signal. The clock generation PLL 25 generates an operation clock, which is used in the AMP & ADC 13, from an internal clock (for example, a clock output from the crystal oscillator 26).

The digital signal processing unit 14 has a clock division unit 28, in addition to the transmission/reception control unit 15, the signal processing unit 16, and the gain control unit 17, described previously. The clock division unit 28 divides a clock output from the crystal oscillator 26 and generates an operation clock that is used in the digital signal processing unit 14.

The power down control unit 21 performs power down control of the AMP (amplifier) 22, the ADC 23, the LVDS (Low Voltage Differential Signaling) 24, the clock generation PLL 25, and the clock control unit 27. Further, in the digital signal processing unit 14, power down is carried out by stopping the clock supply.

As illustrated in FIG. 4, the power down control unit 21 outputs a power down control signal in correspondence to the following control units.
pulser & switch 12: power down by enable signal (PS_EN) control
AMP 22: power down by enable signal (AMP_EN) control
ADC 23: power down by enable signal (ADC_EN) control
LVDS 24: power down by enable signal (LVDS_EN) control
PLL 25: power down by enable signal (PLL_EN) control
digital signal processing unit 14: power down by clock control to the digital signal processing unit 14 with an enable signal (CLK_EN). At the normal time, the clock from the crystal oscillator 26 is connected and at the time of power down, the clock is masked by the clock control unit 27.

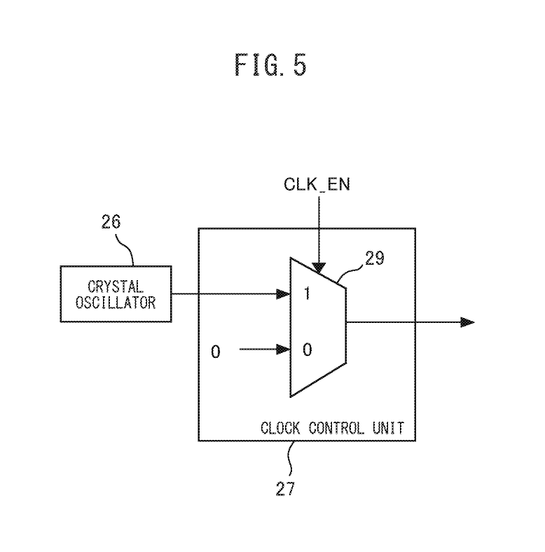

FIG. 5 is a diagram illustrating a configuration of the clock control unit.

The clock control unit 27 has a selector 29 that selects one of the clock from the crystal oscillator and the fixed value (here, 0) in accordance with the CLK_EN signal.

As described above, powered down blocks are freely selected, and it is desirable to select a block by considering not only the power consumption but also the time taken to resume the operating state from the power down state.

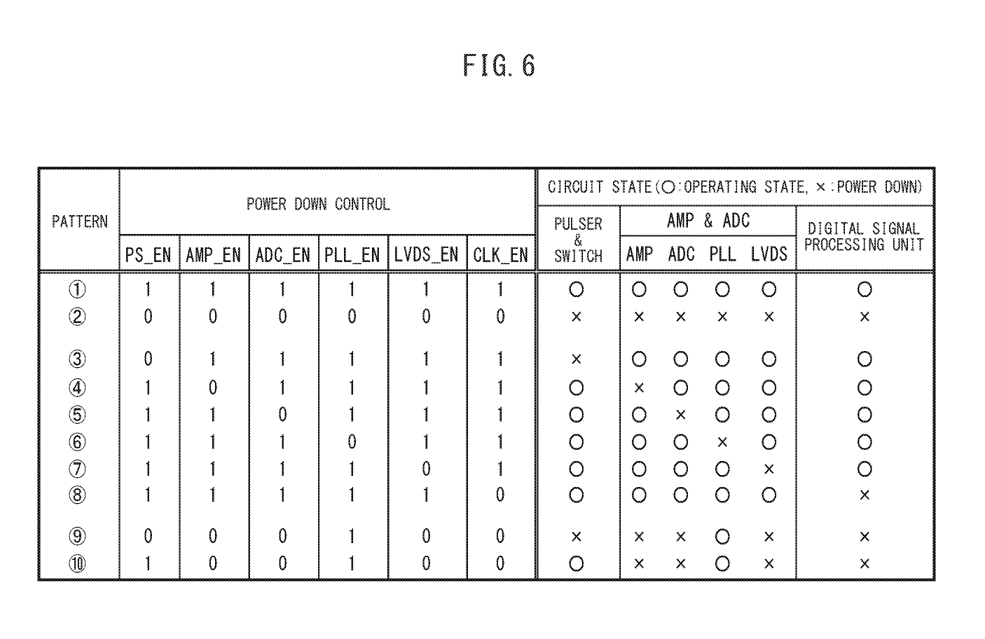

FIG. 6 is a diagram showing a table of combination examples of blocks that power down.

Pattern 1 is a combination in which all the blocks are in the operating state, a combination during measurement.

Pattern 2 is a combination in which all the relevant blocks are in the power down state, a combination that can implement the lowest power consumption.

Each of Pattern 3 to 8 describes a circuit state when one of a plurality of power down control signals is enable (power down control signal “0”). From the patterns, it is known that the control signal and the circuit state can be controlled in a one-to-one manner.

Pattern 9 is a combination in which only the power down control signal PLL_EN is disenable (power down control signal “1”) and the other five power down control signals are enable. The clock generation PLL 25 controlled by PLL_EN requires a stability-waiting period of time until the circuit is stable and outputs an operation clock and when the period is problematic, it is desirable not to power down only a block which requires the stability-waiting period.

Pattern 10 is a combination in which the pulser & switch 12 is also not powered down, in addition to the clock generation PLL 25. Once the pulser & switch 12 is brought into the power down state, it is necessary to set again the amount of delay of the transmission pulse and the output channel to the register, and therefore the period of time until the rise lengthens.

The relationship between the length of the period of time until the rise and the degree of a reduction in power consumption is a trade-off relationship, and therefore a combination of powered down units are may be selected in accordance with an item to which priority is given.

As above, the embodiment is explained, but all the examples and conditions described here are described for the purpose of aiding understanding of the invention and the concepts of the invention applied to the art. Particularly, the examples and conditions described are not intended to limit the scope of the invention and the configurations of such examples in the specification do not indicate the superiority and inferiority of the invention. The embodiment of the invention is described in detail, but is should be understood that the various changes, substitutions, and alterations can be made without deviating from the sprit and scope of the invention.

### CITATION LIST

10 wireless probe
11 transducer
12 pulser & switch
13 AMP & ADC
14 digital signal processing unit
15 transmission/reception control unit
16 signal processing unit
17 gain control unit
18 (wireless probe) system control unit
19 communication unit
20 switch
21 power down control unit
22 amplifier
23 ADC
24 LVDS
25 clock generation PLL
26 crystal oscillator
27 clock control unit
28 clock division unit

What is claimed is:

1. An ultrasonic image generation system comprising:
an ultrasonic transducer configured to transmit and receive an ultrasonic signal;
a processor configured to repeat processing to generate an ultrasonic image signal by processing a received signal of the ultrasonic transducer as well as to generate a drive signal that is supplied to the ultrasonic transducer; and
a display configured to repeat displaying an ultrasonic image based on the ultrasonic image signal, to stop updating of a displayed image in response to a stop signal input, and to resume updating of the displayed image in response to a start signal input, wherein
the processor stops at least part of an operation in response to the stop signal input and resumes the stopped operation in response to the start signal input.

2. The ultrasonic image generation system according to claim 1, comprising:
a probe including the ultrasonic transducer and the processor; and
a tablet including the display and independent of the probe, wherein
the probe and the tablet each include a communicator configured to wirelessly communicate with each other, and
the probe is driven by a battery.

3. The ultrasonic image generation system according to claim 2, wherein
the stop signal input is an input by an operation of a switch provided to the probe or by a touch screen operation of the tablet, and
one of the probe and the tablet, which has detected the stop signal input, notifies the other of the stop signal input via the communicator.

4. The ultrasonic image generation system according to claim 3, wherein
the probe enters a power source off state when the switch has been operated for a predetermined time or longer.
